(19) United States
(12) Patent Application Publication
(10) Pub. No.: US 2003/0162225 A1
(43) Pub. Date: Aug. 28, 2003

(54) Ligands specific for an isoform of the prion protein

(76) Inventors: William Siward James, Oxford (GB); James Hope, Newbury (GB); Abdessamad Tahiri-Alaoui, Oxford (GB)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/295,798

(22) Filed: Nov. 15, 2002

Related U.S. Application Data
(63) Continuation of application No. PCT/GB01/02228, filed on May 18, 2001.

(30) Foreign Application Priority Data
May 18, 2000 (GB) 0012054.3

(51) Int. Cl. G01N 33/53; C07K 16/18

(52) U.S. Cl. 435/7.1; 530/387.1

(57) ABSTRACT

Prion protein, PrP, ligands are provided, especially protease resistant and nuclease resistant ligands. Ligands selective for isoforms such as PrP^{SC} can be prepared. In a related aspect, the PrP ligands are used in diagnostic tests for PrP. The ligands also have potential for a role in the development of therapeutic methods for treatment of TSEs.

The present invention relates to ligands. More particularly the invention relates to ligands for prion proteins.

### BACKGROUND OF THE INVENTION

Transmissible spongiform encephalopathies (TSEs), which include Creutzfeldt-Jacob disease (CJD), variant CJD (vCJD), bovine spongiform encephalopathy (BSE) and scrapie, are characterized by the accumulation of aggregates of the abnormal prion protein (PrP^{SC}) in the brain and other infected tissues^{1,2}. The normal form, PrP^{C}, which is dominated by α-helices towards the C-terminus³⁻⁵, is most abundant in the central nervous system but its physiological function is unknown.

The accumulation of the β-structure rich isoform, PrP^{SC}, is now widely believed to result from the ability of this isoform to stabilize thermodynamically unfavorable, similarly folded forms during the folding of cellular PrP^{C}. Although native PrP^{C} can show distinct intermediates during unfolding^{6,7} it appears to fold from the fully denatured form very rapidly and without intermediates⁸, conforming to the “extended nucleus” model for two-state protein folding⁹, rather than the more usual idea of secondary structure frameworks. This interpretation leads to the notion that PrP might fold by a nucleation-condensation mechanism, whose outcome could, in principle, be diverted by the presence of an alternatively structured nucleation seed.

The structural transitions involved in this process are difficult to study and so the development of selective ligands for the different isoforms would provide invaluable tools for studying prion disease pathogenesis. In addition, reagents that were able to bind PrP^{SC} with high affinity but were less able to bind PrP^{C} might enable one to develop sensitive methods of early diagnosis.

Conventional antibody technology has not yet produced a PrP ligand of the appropriate selectivity, despite strenuous efforts using PrP knockout mice as recipients¹⁰ and phage-display technology¹¹. A monoclonal antibody, 15B3, described by Oersch¹² has not yet been made widely available and so must still be considered unproven. More fundamentally, anti-PrP antibodies are sensitive to the proteases that are often used to remove PrPC from PrPSc-containing samples¹³.

As an alternative approach, the use of nucleic acid ligands, known as aptamers, derived by in vitro selection from synthetic oligonucleotide libraries, is possible in order to develop protease-resistant reagents with appropriate selectivity. RNA aptamers have been isolated against the protease-sensitive, N-terminus of PrP^{14, 14a} but these do not discriminate between PrP^{C} and PrP^{SC} and are very sensitive to nucleases.

### SUMMARY OF THE INVENTION

The present invention provides PrP ligands. In particular, the invention provides protease resistant PrP ligands. Furthermore, the invention can provide nuclease resistant ligands.

In a related aspect, the PrP ligands are used in diagnostic tests for PrP. The ligands of this invention have potential for a role in the development of therapeutic methods for treatment of TSEs.

Preferred Embodiments

In one preferred aspect, the ligands of this invention are selective for PrP and do not have a general ability to bind to proteins. Typically the ligands are not species-specific, and are applicable in species such as humans, cattle and sheep, though specificity can be introduced if desired.

The affinity constant for binding to PrP is suitably in the range of 10 pM to 10 μM, preferably 1 to 10,000 nM, more preferably 10 to 1,000 nM. Selective binding for a PrP isoform is preferred, with selective binding to PrP^{Sc} being especially preferred. In this case the ratio of affinity constants for PrP^{C}:PrP^{Sc} is ordinarily at least 2:1, preferably at least 5:1. With a ratio of 10:1, the ligand has an affinity to PrP^{Sc} which is 10 times that for PrP^{C}, though higher values up to 100 or more may be desirable. Illustratively, the affinity constant for binding to PrP^{Sc} is in the range 20 to 100, and for PrP^{C} is in the range 200 to 1000 nM.

The invention significantly provides nuclease-resistant, protease-resistant ligands for PrP that have selectivity towards the disease isoform of the protein. Such ligands are conformationally selective and can be used to identify disease material under realistic working conditions.

Thus, the differential binding characteristics of the preferred ligands enables a diagnostic test for TSEs to be devised. Accordingly, the invention provides a method of diagnosing a disease such as CJD, vCJD, BSE or scrapie. The diagnosis can be employed at a pre-clinical stage, preferably as a non-invasive procedure, for example as part of a screening program.

A diagnostic method of this invention might comprise preparing a PrP-enriched sample, for example by crude fractionation, and incubating with the ligand in the presence of a protease. Binding of ligand to PrP can be detected in a manner appropriate to the ligand, and may involve labelling. In a preferred method, the ligand-PrP complex can be detected by gel electrophoresis.

The present invention aldo provides a method of preferentially binding a PrP in a biological liquid. Particularly, the present invention provides a method of preferentially binding a predetermined PrP isoform in a biological composition. In a particular embodiment, the present invention provides a method of preferentially binding a PrP^{SC} in a biological composition. The method of the invention comprises incubating a ligand of the invention with a biological composition comprising or believed to comprise a PrP under conditions appropriate for binding of the ligand to the prior protein. Optionally, the binding of the ligand to the prion protein and/or the absence of binding of proteins other than the desired PrP to the ligand may be detected.

A biological composition as used herein may comprise proteins, cells, organ tissue such as tissue from brain, tonsils, ileum, cortex, dura mater, lymph nodes, nerve cells, spleen, muscle cells, placenta, pancreas, bone marrow and/or body fluid, for example blood, cerebrospinal fluid, milk, saliva or semen.

We also provide pharmaceutical compositions containing a PrP^{Sc}-selective ligand of this invention with a pharmaceutically acceptable carrier or diluent.

Specific Embodiments

In a specific embodiment, the invention provides a nuclease-resistant PrP aptamer ligand. The aptamer can comprise 10 to 50 or more nucleotides. The aptamer ligand is suitably a 2′-F-substituted nucleic acid, though other approaches can be used to impart nuclease stability.

In the accompanying FIG. 6, we give the sequence of clones encoding ligands for PrP. The different sequences of FIG. 6 are as follows:

CUUUC CUAGCGCACAUGCGCACCUCUAUGCGUA AUACGAACGUUGA

CG

CUUUC CUAACGCACAUGCGCACCUCUAUGCGUA AUACGAACGUUGG

CG

CGUUC CUAACGCACAUGCGCACCUCUAUGCGUA AUACGAACGUUGG

CG

CUCUC CUAACGCACAUGCGCACCUCUAUGCGUA AUACGAACGUUGG

CG

CUUUC CUAACGCACACGCGCACCUCUAUGCGUA AUACGAACGUUGG

CG

CUGUC CUAACGCACACGCGCACCUCUAUGCGUA AUACGAACGUUGG

CG

CUUUC CUAGCGCACAUGCGCACCUCUAUGCGUA AUACGAACGUUGG

CG

CUUUCGCUAGCGCACAUGCGCACCUCUAUGCGCAUAUACGAACGUUGG

CG

CUUUC CUAGCGCACACGCGCACCUCUAUGCGUA AUACGAACGUUGG

CG

CUUCC CUAGCGCACAUGCGUACCUCUAUGCGUA AUACGAACGUUGG

CG

CUUUG CUAGCGCACAUGCGCACCUCUAUGCGUA GUACGAACGUUGG

CG

CUUUC CUAGCGCACAUGCGCACCUCUAUGCGUA AUACGAACGUCGG

CG

CUCUC CUAGUGCACAUGCGCACCUCUAUGCGUA AUACGAACGUUGG

CG

CUUUC CUAGCGCAUAUGCGCACCUCUAUGCGUA AUACGAACGUUGG

CG

CUUUC CUAGCGCAUAUGCGCACCUAUAUGCGUA AUACGAACGUUGG

CG

CUUUC CUAGCGCAUAUGCGCACCUCUAUGCGUA AUACGAACGUCGG

CG

CUUUG CUAGCGCACAUGCUCACCUCUAUGCGUA AUACGAACGUUGA

CG

CUUUC CUAGCGCACAUGCGCACCUCUAUGCGUA AUACGAACGUAGA

CG

CUUUC CUAGCGCACAUGCGCACCUCUACGCGUA AUACGAACGUUGA

CG

CUUUC CUAGCGCACAUGCGCACUUCUAUGCGUA AUACGAACGUUGG

CG

CUUUC CUAGCGCACAUGCGCACUUCUAUGCGUA AUACGAACGUUGG

CG

CUUUC CUAAUGCACAUGCGCACCUCUAUGCGUA AUACGAACGUUGA

CG

CUUUC CUAACGCAUAUGCGCACCUCUAUGCGUA AUACGAACGUUGA

CG

CUUUC CUAGUGCAUAUGUGCACCUCUAUGCGUA AUACGAACGUUGG

CG

CUUUC CUAGUGCAUAUGUGCACCUCUAUGCGUA AUACGAACGUUGG

CG

CUUUC CUAACGCAUAUGUGCACCUCUAUGCGUA AUACGAACGUUGA

CG

CUUUC CUAACGCACAUGCGCACCUCUAUGCGUA AUACGAACAGUGA

GA

GGUUUCGACCA GCACCUUGACCGAUUCCACAGCUCUGCGGGAGA
CUCCUCUA GCACCAUAUCCAAGCUACAACUUCACAACGACUCGGC

C

CUACGAACUCAUGACACAAGGAUGCAAUCUCAUCCCGCCAGC

CC

CUACGUUCCUUAUCCUCCCUUCAGGAACCUGUACACCACAUU

GC

UAUCAACAUUAGGGCUCCUUGGGGACCAGCGUCUCCUUGCAGCCCCGA
GCUGACCACCGCCAACGCAACCUCCAAUGACUUGGAUCACCUAGACG

AU

In an aspect of this invention, we provide aptamers with such a sequence as listed above or as seen in FIG. 6, and variants thereof.

The variant aptamer ligands of this invention include:

(a) aptamers with at least 15, 18, 20, 25, 30, 35, 40 or more nucleotides in common with a sequence of FIG. 6, particularly aptamer ligands with 15, 18, 20, 25, 30, 35, 40 or more consecutive nucleotides identical to 15, 18, 20, 25, 30, 35, 40 or more consecutive nucleotides of a sequence of FIG. 6, respectively; and

(b) aptamers which are at least 80% identical with a sequence of FIG. 6 or with an aptamer (a).

In respect of (b), there is preferably at least 90% identity, such as at least 95% or at least 98% identity. Sequence identity is suitably determined by a computer programme, though other methods are available. We prefer that identity is assessed using the BestFit software from the Wisconsin/Oxford Molecular GCG package.

One example of a typical motif believed to bring about common PrP binding is:

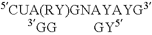

Aptamers with this motif are preferred, especially monoclonal aptamers.

Methods for preparing aptamer ligands are provided by this invention.

In a further, related aspect of this invention, we provide a procedure for preparing a full length PrP in β-form.

In one embodiment, we provide ligands that can discriminate between normal and disease isoforms of the prion protein (PrP). In particular, we have isolated 2′-F nucleic acid ligands, or aptamers, to the abnormal PrP isoform derived from scrapie-infected hamster brain. The aptamers are highly specific to PrP and bind to the protein from several species, including humans, cattle, sheep, hamster and mouse. They have affinities in the range 10⁻⁷ M and have 10-20-fold higher affinity for a β-isoform than the normal, α-isoform of recombinant PrP. This property can be used to identify the presence of abnormal PrP in samples of infected tissue. These aptamers might therefore be used to develop a sensitive assay for material infected with the agents of BSE, scrapie and CJD. Furthermore, we show that one of our aptamers, aptamer 93, can inhibit PrP conversion in vitro.

### EXAMPLE

The present invention is illustrated by the following example based on our experimental work.

We describe the isolation of aptamers based on nuclease-resistant, 2′ F chemistry^{15,16} some of which show substantial selectivity in favor of the PrP^{Sc} isoform. These novel ligands will be useful in the development of simple diagnostic tests for TSEs and in the analysis of TSE pathogenesis.

Materials and Methods

Oligonucleotides

All oligonucleotides used in this study (see Table 1) were synthesized by Genosys (Cambridge, UK).

TABLE 1

| Name | sequence |
|---|---|
| Li- | AATTAACCCT CACTAAAGGG AACTGTTGTG AGTCTCATGT |
| brary | CGAA(N)₅₀ TTGAGCGTCT AGTCTTGTCT |
| T3 | AATTAACCCT CACTAAAGGG AACTGTTGTG AGTGTCATGT |
| selex | CGAA |
| T7 | TAATACGACT CACTATAGGG AGACAAGACT AGACGCTCAA |
| selex | |
| Eco RI | CCGGAATTCC GGAATTAACC CTCACTAAAG GGAACTG |
| selex | |
| Sma I | TCCCCCGGGG GATAATACGA CTCACTATAG GGAGAC |
| selex | |
| For- | GCACCCCAGG CTTTACACTT TATGC |
| ward | |
| Re- | CAGGGTTTTC CCAGTCACGA CGTTG |
| verse | |
| 3′, | AATTAACCCT CAC |
| 13-mer | |

In vitro Selection

The library oligonucleotide pool (see Table 1) comprising a region of 50 randomized nucleotides flanked by T3 and T7 transcriptional promoter sequences was converted into double stranded template following a protocol previously described by Tuerk¹⁷. All RNAs used for in vitro selection were produced by in vitro transcription with T7 RNA polymerase in presence of 2′-fluoro modified pyrimidine nucleotide triphosphates (TriLink BioTechnologies, Inc., San Diego), together with unmodified purine ribonucleotides in an optimized transcription buffer¹⁸. The 2′-F RNA transcripts were purified by electrophoresis on a 10% (w/v) denaturing polyacrylamide get in TBE buffer.

The pool of 2′-F RNA was heat denatured for 2 minutes at 95° C. in deionized and filter-sterilized water, refolded for 10 minutes at room temperature in HMKN buffer (20 mM Hepes pH 7.2, 10 mM MgCl₂ and 50 mM KCl, 100 mM NaCl), before being used for the selection process. The refolded 2′-F RNA pool (5 nmol) was incubated with scrapie associated fibrils (SAF) purified from approximately one-half of a hamster brain, prepared as described below. Before each round of selection an aliquot of SAF was sonicated in a cup-horn probe with three pulses of one minute each with an amplitude set at 40 and an output of 20 W. The binding reaction was done at room temperature in HMKN buffer for four hours. After partitioning the binding reaction was centrifuged for one hour at 25,000×g at 10° C. The amount of unbound 2′ F-RNA in this first supernatant was stored at −20° C.

In order to remove non-specifically bound 2′ F-RNA, the pellet containing 2′ F-RNA-SAF complex was washed three times with 100 μl HMKN buffer. The supernatants from each wash were pooled with the first supernatant and the amount of unbound 2′ F-RNA was determined by spectrophotometer (GeneQuant, Pharmacia UK). To recover a cDNA library enriched for aptamer-encoding sequences, the pellet containing bound 2′ F-RNA was incubated with Tth DNA polymerase, T7 selex and T3 selex primers at 70° C. for 20 minutes followed by PCR amplification following the protocol provided by the supplier (Promega WI, USA).

Preparation of Scrapie Associated Fibrils

Scrapie-associated fibrils (SAF) were prepared from the brains of hamsters that were infected with the 263 K strain of scrapie¹⁹. SAF were prepared without proteinase K treatment essentially as described by Hope et al.¹ The final pellet (P285) was washed several times in water to remove traces of sarcosinate, before being resuspended in HMKN buffer pH7.2 containing 0.02% azide and stored at +4° C.

Production of Recombinant Bovine, Murine and Sheep PrP Proteins

DNA sequences encoding methionine-initiated mature-length PrP proteins from cattle (6 octarepeat allele), mouse (S7 allele) and sheep (ARQ allele) were obtained by PCR amplification of genomic DNA and inserted as BgLII-EcoRI restriction fragments into expression plasmid pMG939²⁰ and amplified in E. coli K12 1B392.pACYRIL, which overexpresses rare arginine, isoleucine and leucine tRNAs. Cultures were grown to saturation in Terrific Broth containing 100 μg/ml ampicillin and 15 μg/ml chloramphenicol at 30° C. then diluted 400-fold. In late log phase, expression of PrP was induced by raising the temperature from to 45° C. for 10 min, followed by incubation at 42° C. for 5 h.

The cells were then harvested by centrifugation at 10,000×g for 15 min. The pellet was resuspended in ice-cold lysis buffer (50 mM Tris-HCl pH 8.0, 100 mM NaCl, 2 mM PMSF, 10 μg/ml lysozyme, 10 μg/ml DNase I, 1 mg/ml sodium deoxycholate) and incubated for 30 min at 37° C. The solution was then centrifuged at 10,000×g for 30 min, and the supernatant discarded. The pellet was washed twice by resuspension in lysis buffer with centrifugation at 10,000×g for 10 min between each wash. Proteins in the pellet were dissolved by suspending it in buffer A (100 mM sodium phosphate, 10 mM Tris pH 8.0, 8 M urea and 10 mM 2-mercapthoethanol) and incubating for 30 min with gentle mixing. Cell debris and insoluble material were removed by centrifugation at 15,000×g for 15 min.

The supernatant was loaded onto a Ni-NTA-Sepharose column (QIAGEN Ltd. Dorking UK Q) pre-equilibrated with buffer A. After washing the column with the same buffer, bound proteins were eluted with buffer B (100 mM sodium phosphate, 10 mM Tris pH 4.5, 8 M urea and 10 mM 2-mercapthoethanol) as recommended by the supplier. For further purification the eluate from the column was diluted 1:2 with buffer C (50 mM Hepes pH 8.0, 8M urea and 10 mM 2-mercapthoethanol) and loaded onto cation exchange chromatography column, SP-Sepharose (Amersham-Pharmacia Biotech). Recombinant PrP was eluted with buffer C supplemented with 1.5 M NaCl. Eluted fractions of recombinant PrP were pooled and disulfide bonds were oxidized by stirring overnight in a 2:1 molar excess of CuCl₂. After oxidation, the protein solution was dialysed against 50 mM Na-acetate pH 5.5, 1 mM EDTA, with several changes of buffer.

Finally, the recombinant PrP was applied onto a size-exclusion chromatography column (Superdex 75 HR 10/30, Amersham-Pharmacia Biotech) equilibrated and eluted with 50 mM Na-acetate pH 5.5.

Cloning and Sequencing of Monoclonal Aptamers

The pool of 2c-F RNA from the seventh round of in vitro selection was reverse transcribed and PCR amplified with EcoR I selex and Sma I selex primers (see Table 1). The resulting PcR product was digested with EcoR I and Sma I, subcloned into EcorRI-cut, SmaI-cut and dephosphorylated pUC18. After ligation and transformation, plasmid DNA was prepared from fifty insert-positive bacterial colonies using QIAGEN resin (QIAGEN Ltd. Dorking. UK) and used as template for sequencing in both directions with the forward and reverse primers (see Table 1) in the presence of PRISM™BigDye™ cycle sequencing ready reaction kit from ABI (Perkin-Elmer). The resulting sequences were compared to each other and aligned using ClustalX (version 1.64B). Four representative 2′-F RNA monoclonal aptamers were selected for further analysis; these were aptainers 73, 76, 90 and 93.

5′ End Labeling of Monoclonal Aptamers

PCR-amplified templates for monoclonal aptamers 73, 76, 90 and 93 were in vitro transcribed as described above. The reactions were incubated overnight at 37° C., RNase free DNase I (Sigma) was added to remove the DNA template, and the reactions were quenched by extracting with an equal volume of phenol-chloroform-isoamylalcohol pH 4.7 (Sigma). The nucleic acids in the aqueous phase were precipitated by adding 0.1 volume of 3 M sodium acetate (pH 5.2) and 2.5 volumes of ethanol. The precipitate was resuspended in formamide stop buffer and purified on a 10% denaturing polyacrylamide gel in TBE buffer. The product band was excised, eluted overnight in 0.5M ammonium acetate, 1 mM EDTA pH6.5 and extracted with an equal volume of phenol/chloroform. The RNA was precipitated with ethanol and washed once with 70% ethanol and then dissolved in water.

To label 2′-F RNA monoclonal aptamers at the 5′ terminus, transcripts were dephosphorylated using bacterial alkaline phosphatase (Pharmacia-Amersham Biotech), incubated in presence of [y-³²P] ATP (Pharmacia-Amersham Biotech), T4 polynucleotide kinase and T4 polynucleotide kinase buffer supplied with the enzyme (Boehringer-Mannheim, GmbH) at 37° C. for one hour. The reaction was terminated by adding an equal volume of formamide stop buffer and resolved on a 10% denaturing polyacrylamide gel in TBE buffer. Labeled 2′-F RNA monoclonal aptamers were visualized by autoradiography, excised from the gel, cluted and precipitated as described above. The purified 2′-F RNA monoclonal aptamers were dissolved in water, quantified by Cérênkov counting and used for gel mobility shift, footprinting and structural analysis.

Affinity and Specificity of Monoclonal Aptamers for Recombinant Bovine PrP

The complex between aptamers and α-PrP or β-PrP was observed as a mobility shift in non-denaturing 0.7% agarose gel in 0.5× TBE. A constant concentration of labeled aptamer (5000 cpm) was incubated with various concentrations of protein (60, 90, 120, 150, 180, 240, 300, 360, 480, 600, 720, 840, 960 and 1080 nM) in 20 mM Hepes pH7.2 for α-PrP or 20 mM Na-acetate pH 5.2 for β-PrP. Both buffers contained 100 mM NaCl, 10 mM MgCl2, 50 mM KCl, 0.06% Nonidet P40 and 0.03 mg/ml of tRNA. Aptamers used for each experiment were heated to 95° C. for one minute in water and cooled at room temperature for 10 minutes in HMKN buffer prior to adding protein. Reaction volumes of 30 μl were incubated for one hour at room temperature before adding 3 μl of loading buffer (50% glycerol, 0.25% bromophenol blue, 0.25% xylene cyanol). The samples were immediately loaded onto 0.7% agarose gel in 0.5× TBE and electrophoresed at 6 V/cm for 90 minutes. After electrophoresis was completed, the gel was vacuum-blotted onto Nylon membrane using Model 785 (BioRad) at 5-7 inches Hg for 40 minutes in 10×SSC.

Gel mobility shifts were imaged using a Molecular Dynamics Storm 840 for quantitation. The binding data were analysed with GraphPad PRISM and fit by non-linear regression to a hyperbolic function.

Nuclease Mapping of Monoclonal 2′-F Aptamers and Footprinting

We used enzymatic probing to determine the secondary structure of 2′-F aptamers. End-labeled aptamers (see above) were heated to 95° C. for one minute in water and allowed to cool to room temperature for 10 minutes in HMKN buffer. Digestion with ribonucleases T1, V1 or S1 (Amersham Pharmacia Biotech) was in 20 μl HMKN buffer²¹. The reaction mixtures contained 1 μg of tRNA, 5′-end labeled 2′-F aptamers (50 000 Cérênkov c.p.m.) and 1 μl of the appropriate enzyme and the reaction was allowed to incubate at 20° C. for 5 minutes. The following amounts of RNases were added: 1 ×10⁻² units of T1, 7×10⁻² units of Vi and 21 units of S1. For footprinting the complex between 2′-F aptamer/recPrP was first allowed to form, following the conditions described for gel shift mobility assays, before carrying out the RNase mapping. Reactions were stopped by extraction with phenol and precipitation of the 2′-F RNA with ethanol. The pellets were washed with 80% ethanol and vacuum-dried. The 2′-F RNA fragments were then sized by electrophoresis on a denaturing 15% (w/v) polyacrylamide gel followed by autoradiography. A partial alkaline hydrolysis ladder of 2′-F aptamers²² was run in parallel with a sequencing reaction with RNase T1 ladder giving the position of the G residues.

Chemical Probing with DMS and CMCT

Chemical probing was done under native conditions, essentially as described²³⁻²⁵. Reaction mixtures of 20 μl contained the appropriate buffer (HMKN buffer pH 7.2 for DMS or 50 mM sodium borate pH 8.0 for CMCT modifications), 2 μg tRNA and 0.1 μg of 2′-F aptamer that had been refolded as described before. To initiate the reactions, 1 μl of DMS (1:8 dilution in ethanol) or 1 μl of CMCT (40 mg/ml in water) were added. The reactions were incubated at 20° C. for 5 minutes (DMS) or 20 minutes (CMCT). Immediately afterward, 2′-F aptamers were precipitated with ethanol. The pellets were dried and dissolved in water. Unmodified 2′-F aptamer controls in the absence of DMS and CMCT were processed in parallel.

Detection of Modified Bases by Primer Extension

Reverse transcription reactions were done essentially as described²¹. The 3′, 13-mer oligonucleotide (see Table 1) was 5′-labelled in presence of [γ-³²P] ATP and T4 polynucleotide kinase as described above and purified from the excess of radioactive ATP by polyacrylamide gel electrophoresis. Modified and control 2′-F aptamers (above) were incubated with primer DNA (50,000 c.p.m.) in hybridization buffer (50 mM Tris-HCl, pH 8.5, 6 mM MgCl₂, 40 mM KCl) at 65° C. for 5 minutes in a final volume of 10 μl and then cooled to room temperature. Elongation was done in 15 μl at 37° C. for 30 minutes in the presence of 2.5 mM each of DATP, dCTP, dGTP and dTTP, and 2 units of avian myeloblastosis reverse transcriptase. Sequencing of the unmodified 2′-F aptamers was done as described²⁶.

Preparation of β-Rich Form of the Full Length Recombinant Bovine PrP

Full-length recombinant bovine PrP in the oxidized α form was converted to the β form largely as described²⁷. Circular dichroism (CD) was used to assess the folding of both α and β form of PrP. Far UV CD spectra were recorded at a protein concentration of 75 μM between 190 and 250 nm at 25° C. in a 0.01-cm path length cuvette. The buffers used were 10 mM Tris-acetate pH 5.0 for β-PrP, and 50 mM Na-acetate pH5.5 containing 1 mM EDTA for α-PrP.

The electrophoretic mobility of α and β form of PrP was analysed in low pH discontinuous native 15% polyacrylamide gel²⁸.

Preparation and Analysis of Brain Homogenates

Brain homogenates from humans, PrP knockout mouse (PrP^{0/0}), control hamster and mouse and from scrapie-infected hamster and mouse, 263K and ME7 strains were prepared at 10% (w/v). Brain homogenates from BSE-diseased cattle and from control animals were prepared at 20% (w/v). Brains were homogenized in HMKN buffer containing 0.5% Nonidet P40. Aliquots of the brain homogenates were stored at −80° C. They were used in gel mobility shift assays either as crude homogenate or, after detergent lysis and ultracentriflgation, as the PrP^{5c} fraction, P285¹. In the latter case, 200 mg samples of brain from TSE-infected and control cattle, mouse and hamster, and of PrP^{0/0} null mice were purified. The equivalent of 6.7 mg of rodent or 13.4 mg of bovine brain was then incubated with aptamer 73 together with proteinase K (50 μg/ml) in a total volume of 20 μl and analysed by agarose gel electrophoresis. Parallel samples of the human and animal brain homogenates were analysed by western blotting using monoclonal antibody 6H4. This confirmed the presence of PrP^{Sc} only in the case of individuals with TSE (data not shown).

Results

### FIGURES

FIG. 1. Binding of polyclonal, selected nucleic acids to purified scrapieassociated fibrils (SAF).

Appearance of SAF-binding nucleic acids in sequential rounds of in vitro selection, detected by depletion. From round 3, there was no further increase in the proportion of RNA bound to PrP^{SC}, as detected by depletion of RNA from the supernatant after mixing with insoluble PrP^{Sc}.

FIG. 2. Affinity and specificity of monoclonal aptamers for recombinant bovine PrP

A. Example of band shift affinity analysis of monoclonal aptamers against recombinant bovine PrP. 5000 c.p.m. (about 0.01 pmol) of ³²P-labelled aptamer 73 was mixed with recombinant bovine PrP at concentrations ranging from 60 nM (lane 2) to 1080 nM (lane 15). Lane 1 contains no PrP.

B. Assays of the sort shown in panel A were quantitated by storage phosphor radiography. Squares (+) correspond to aptamer aptamer 73, inverted triangles (▾) to aptamer 76, diamonds (♦) to aptamer 90 and triangles (▴) to aptamer 93.

C. Specificity of PrP-binding aptamer. Band-shifts were performed with ³²P-end-labelled aptamer ap93 and a range of proteins. Lane 1, no protein; lanes 2 and 4, recombinant bovine PrP (500 nM); Lane 3, recombinant sheep PrP(500 nM); lane 5, recombinant mouse PrP (500 nM); lane 6, recombinant human CD4 (500 nM); lane 7, streptavidin (500 nM).

D. Displacement of ap90/PrP complex by unlabelled ap90. ³²P end-labeled aptamer 90 (5000 cpm) was incubated with alpha form of recombinant bovine PrP (720 nM) and increasing concentrations of unlabelled ap90. Lane 1, no competitor. Lane 2, 80 nM; lane 3, 90 nM; lane 4, 100 nM; lane 5, 100 nM; lane 6, 140 nM; lane 7, 150 nM; lane 8, 200 nM unlabelled ap90 competitor.

FIG. 3. In vitro conversion of recombinant bovine PrP to a γ-rich form

A. Circular dichroism of recombinant bovine PrP before (continuous line) and after (dashed line) reduction of disulphides, denaturation with 6M guanidinium and refolding in Tris acetate pH 5.0

B. SDS-PAGE analysis of native, recombinant bovine PrP (lane 2) and three separate batches of β-rich, refolded PrP (lanes 3-5)

C. Native PAGE using the pH 4.4 Reisfield system of native and refolded recombinant bovine PrP. The β-form has a lower mobility, produces a sharper band and stains less well with Coomassie than the α-form. The common low mobility band is probably an oligomer of PrP.

FIG. 4 Affinity of aptamers for β-form bovine PrP

A. The affinity of aptamers for in vitro-refolded, β-rich isoform of PrP was measured by performing gel-retardation assays between 0.01 pmol of ³²P-labelled aptamer and varying concentrations of protein. In this example, the aptamer was aptamer 76.

B. The proportion of aptamer complexed with protein was quantitated by storage phosphor radiography. Squares (+) correspond to aptamer ap73, inverted triangles (▾) to ap76, diamonds (♦) to ap90 and triangles (▴) to ap93.

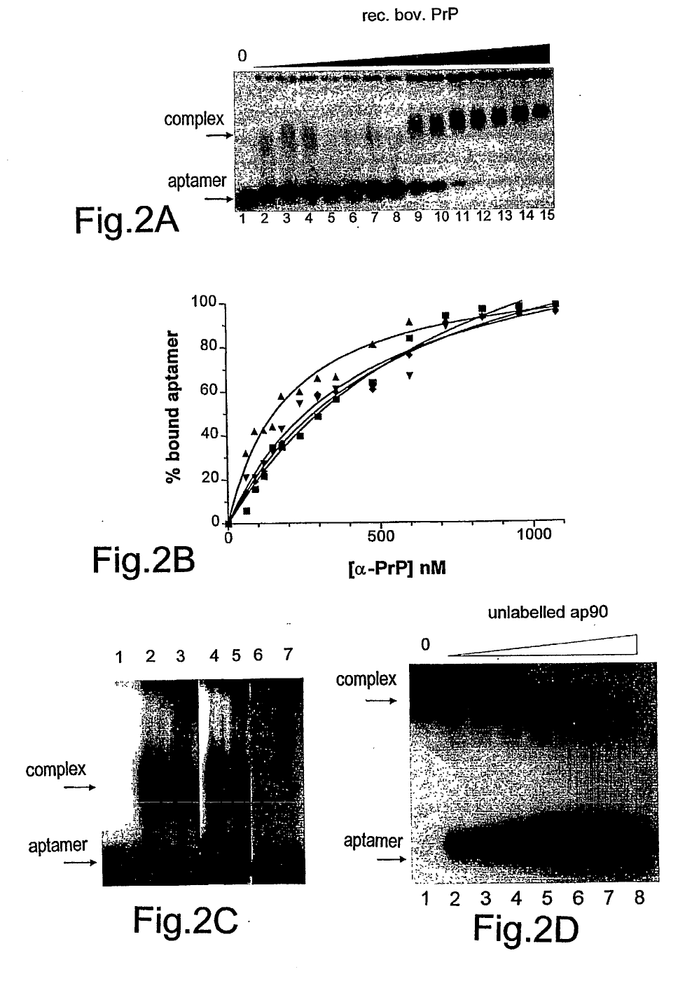

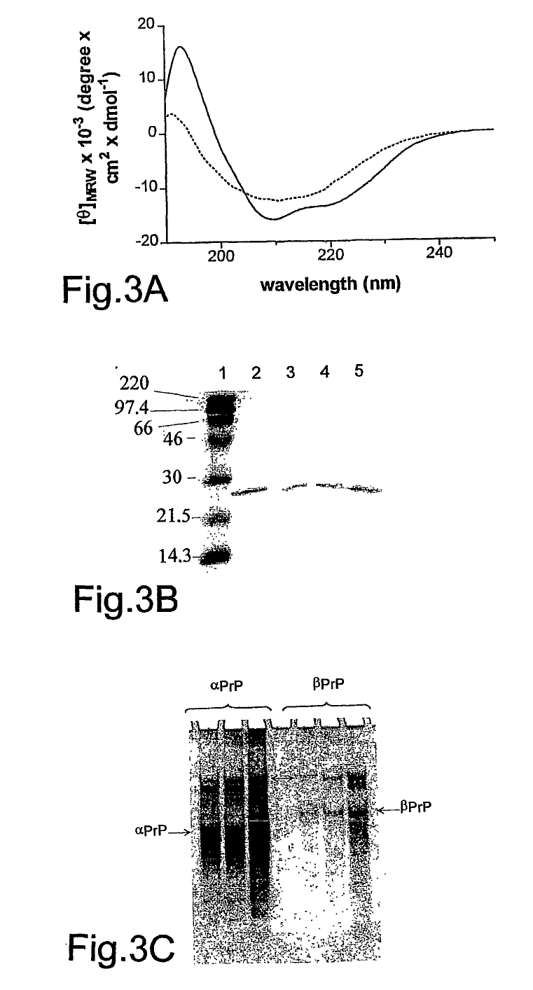

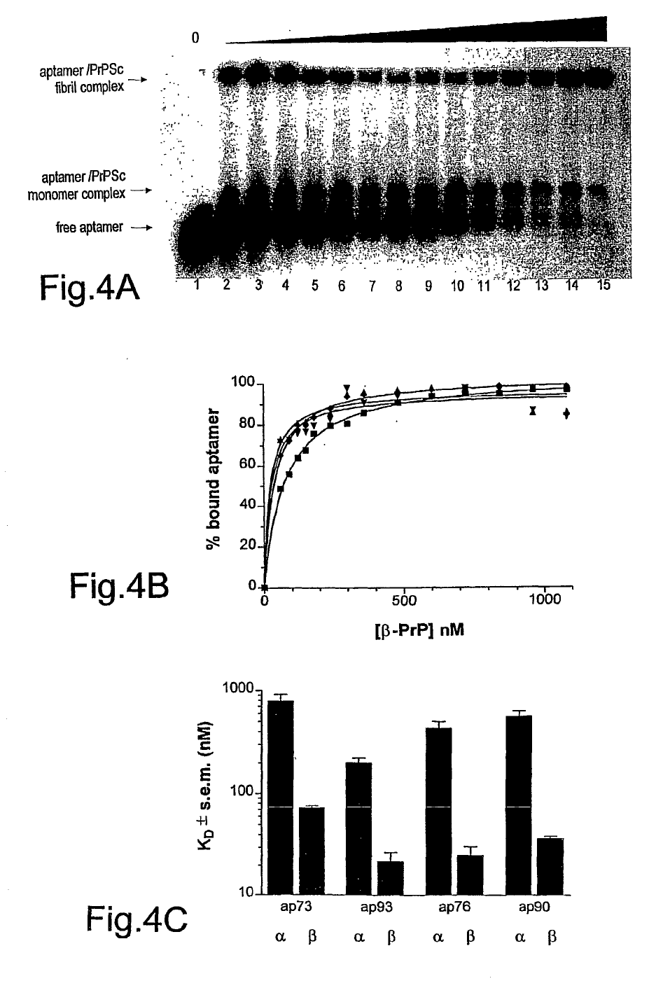

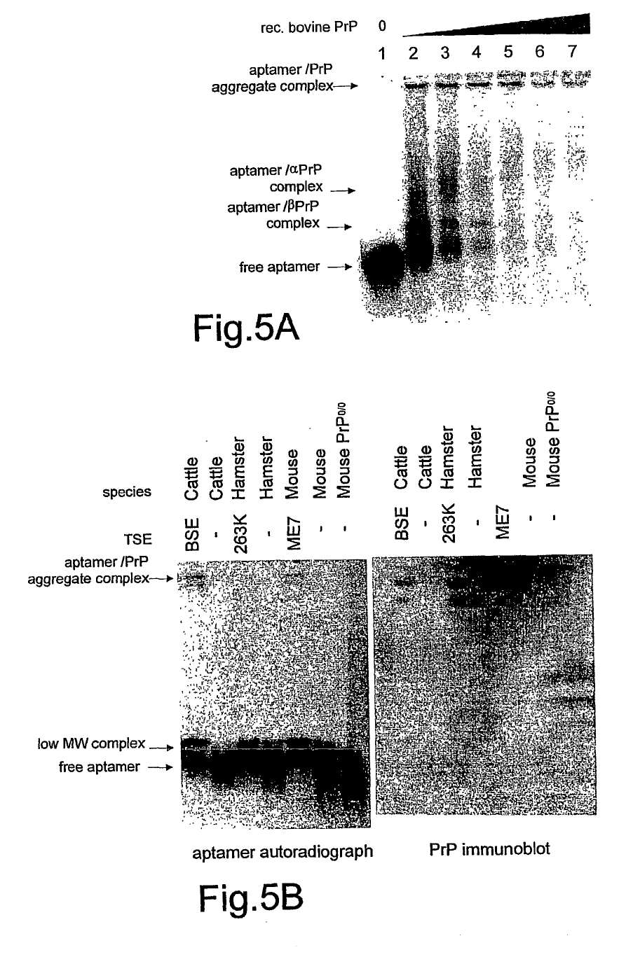

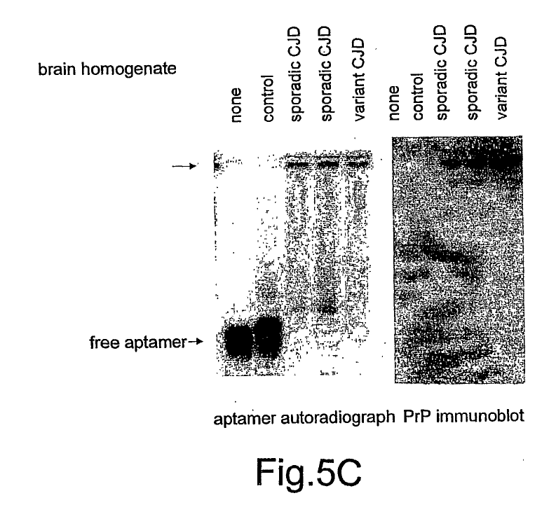

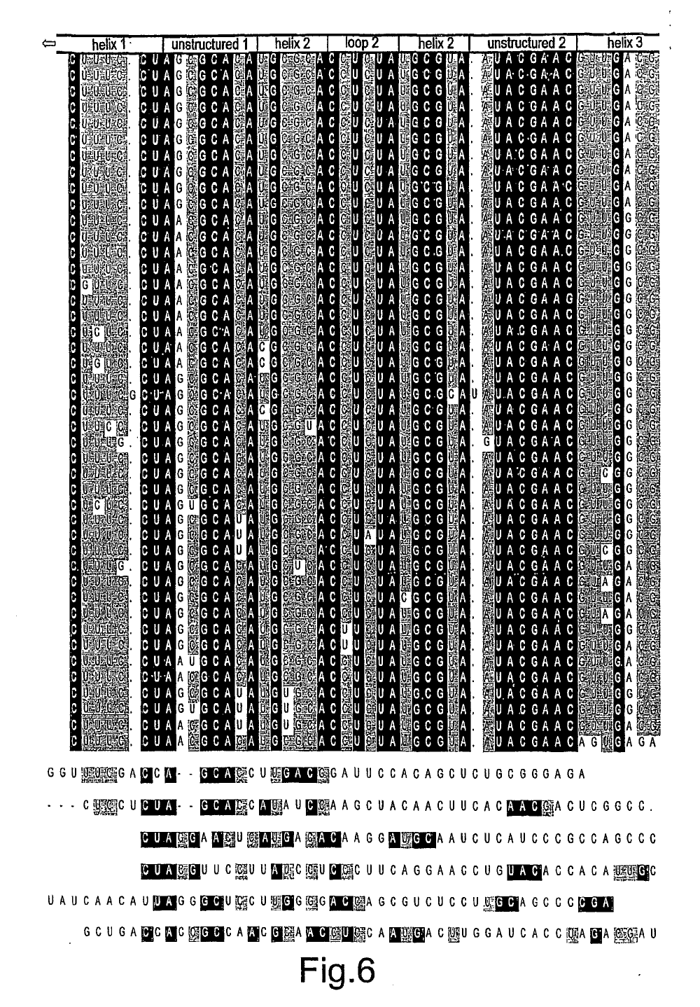

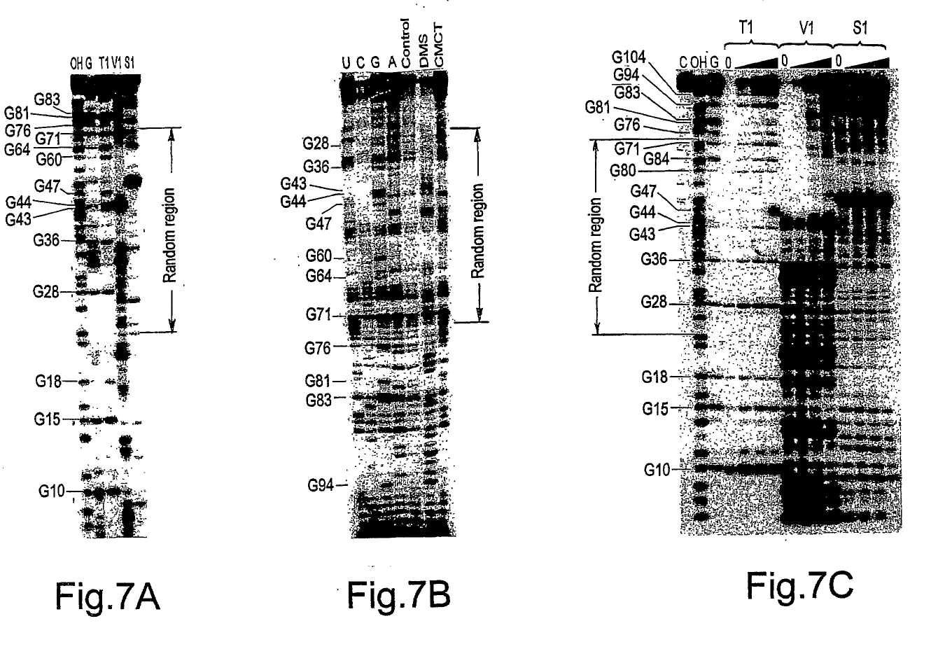

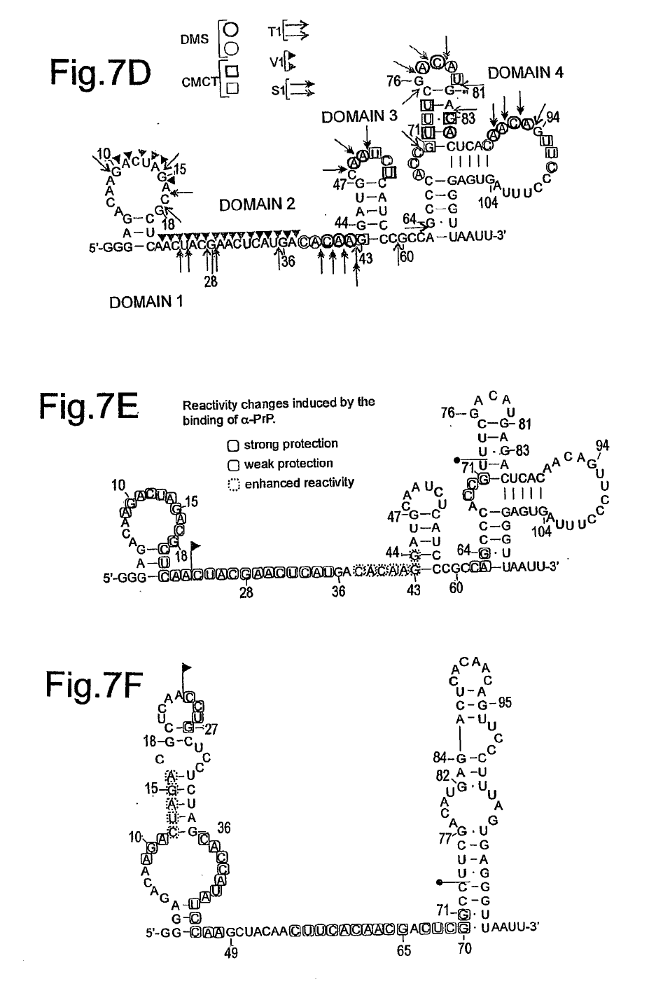

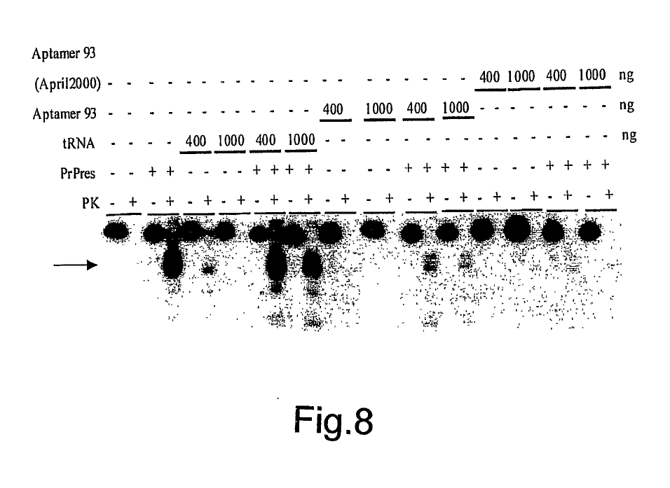

C. C. Comparison of affinity of aptamers for a and β-form PrP. The concentration-dependence of aptamer interaction with α-form bovine PrP (FIG. 2B) and β-form bovine PrP (FIG. 4A) was fitted to a hyperbolic function by non-linear curve fitting. The error bars represent the standard error of the mean of the hyperbolic fit.

FIG. 5 Discrimination between normal and abnormal forms of PrP by aptamers

A. Detection by gel-retardation, of low concentration of β-form PrP in presence of the α isoform. A constant amount of ³²P-labelled PrP aptamer 73 (5000 c.p.m.) was incubated with an equimolar mixture of α and β-form recombinant bovine PrP at final concentration of 0, 60, 120, 150, 180, 240 and 300 nM (lanes 1-7, respectively). Aptamer-PrP complexes were separated from free aptamer by agarose gel electrophoresis.

B. Detection of disease-specific bands corresponding to aptamer/PrP complexes in purified samples of infected and control hamster, cattle and mouse brain. The insoluble fraction of detergent-extracted brain samples were incubated with ³²P-labelled PrP aptainer 73 and then analysed by agarose gel electrophoresis, as described in Methods. The left-hand panel shows the autoradiograph revealing the position of aptamer and aptamer-PrP complexes. The right hand panel shows a parallel immunoblot, using monoclonal anti-PrP antibody 6H4 to detect the presence of PrP and PrP-containing complexes.

C. Detection of disease-specific PrP complexes with aptamer in human brain samples. Samples of cerebral cortex from a normal human (RU97/03, lanes 2 and 7), from two cases of sporadic CDJ (RU991009, lanes 3 and 8; RU97/008 lanes 4 and 9) and from a case of variant Clix (RU98/148, lanes 5 and 10) were homogenized, clarified by low-speed centrifugation, treated (lanes 6-10) or not treated (lanes 1-5) with proteinase K and mixed with ³²P-labelled PrP aptamer 73. High molecular aptamer-PrP complexes were separated from free aptamer by agarose gel electrophoresis. The left-hand panel shows the autoradiograph revealing the position of aptamer and aptainer-PrP complexes. The right hand panel shows a parallel immunoblot, using monoclonal anti-PrP antibody 6H4 to detect the presence of PrP and PrP-containing complexes.

FIG. 6. Sequences of PrP-binding aptamers

The sequences of 25 aptamer clones (randomized regions only) are shown. The majority fall into a closely related group, which are aligned in the figure using ClustelX (1.64B). Dots represent gaps introduced for alignment purposes. Nucleotides shown in white-on-black are absolutely conserved among this group, while those shown in black-on-gray are >75% conserved. Small stretches of homology are apparent between the three orphan aptamers and the consensus of Group I and the nucleotides are laterally displaced and shaded in the figure to highlight these.

FIG. 7. Secondary structure and epitope-mapping of four PrP-binding aptamers

A. Nuclease mapping of aptamer 93 under statistical conditions. Example of an auto radiogram of 15% polyacrylamide gel illustrating the cleavage products of 5′-labeled 2′-fluoro-aptamer 93. Lanes OH and G represent hydroxyl and RNase TI ladders, respectively. The gaps in the hydroxyl ladder indicate the positions of 2′-fLuoro-pyrimidines that are resistant to alkaline hydrolysis. Lane TI, RNase T1 mapping; lane V1, RNase V1 mapping; lane S1, RNase S1 mapping assayed at 20° C. for 5 minutes at pH 7.2 using the following amount of nucleases: 1×10-2 units of T1, 7×10-2 units of V1 and 21 units of S1.

B. Example of chemical probing of aptamer 93 with (DMS), dimethyle sulfate and (CMCT), 1-cyclohexyl-3-(2-morpholinoethyl)carbodiimide metho-p-toluene sulfonate. Auto-radiogram of 10% polyacrylamide gel of primer extension products using 5′-end-labelled oligonucleotide (AATTAACCCTCAC) complementary to the 3′ end of aptamer. 2′-fluoro-aptamer 93 was probed in 1× HMKN buffer pH 7.2 for DMS and in 50 mM sodium borate pH 8.0 containing 10 mM MgCl2 and 50 mM KCl for CMCT. Reactions were carried out at 20° C. for 5 and 20 minutes for DMS and CMCT, respectively. Unmodified (control lane) 2′-fluoro-aptamer 93 was run in parallel to discriminate between stops specifically induced by chemical modifications and those due to the presence of stable secondary structures or spontaneous cleavages. Note that primer extension stops one residue prior to the modified bases, so the bands in the probing lanes are shifted down one residue relative to the corresponding sequencing bands. Lanes U, C, G and A are specific reverse transcription sequencing ladder.

C. Example of an auto radiogram of 18% polyacrylamide gel illustrating the footprinting of recombinant bovine alpha-PrP binding site onto 2′-fluoro-aptamer 93 using nucleases T1, V1 and S1 Lane C, control 5′-end labeled 2′-fluoro aptamer; Lanes OH and G represent hydroxyl and RNase T1 ladders, respectively. The black wedges at the top of the gel indicate the increasing concentrations (0, 120, 360 and 1080 nM) of alpha-PrP.

D. Composite of nuclease cleavages and chemical modifications overlaid on the deduced secondary structure model of 2′-fluoro-aptamer 93. The intensities of cuts/modifications are proportional to the darkness of the symbol.

E. Reactivity changes towards nucleases T1, V1 and S1 induced by the binding of alpha PrP to 2′-fluoro-aptamer 93 overlaid on the proposed secondary structure. Three degrees are distinguished; weak or mild protection against nucleases attack or enhanced reactivity towards nucleases.

F. As in figure (E), but for 2′-fluoro-aptamer 76.

FIG. 8. Inhibition of PrP conversion in vitro by aptamer

Recombinant PrP, tagged with-the epitope for the 3F4 antibody was prepared in alpha helix-rich, native form was incubated in the presence or absence of either PrP-specific aptamer 93 or the non-specific tRNA. This mixture was then incubated in the rpesence or absence of scrapie-associated fibrils derived from infected hamster brain (PrPres) according to the method of [Kocisko, 1995 #556], under which conditions the recombinant protein normally acquires the protease resistance properties of the infectious Prpres. This conversion process was assessed by incubating the mixtures in the presence or absence of proteinase K (PK) and then subjecting them to SDS-P AGE. The appearance of a band (marked by the arrow) in the lanes corresponding to PK-treated samples is indicative of conversion. In the experiment shown, it is evident that the conversion process is substantially inhibited by aptamer 93 but not by tRNA.

Construction of a 2′ F RNA Library and Enrichment for PrP^{Sc}-Binding Sequences

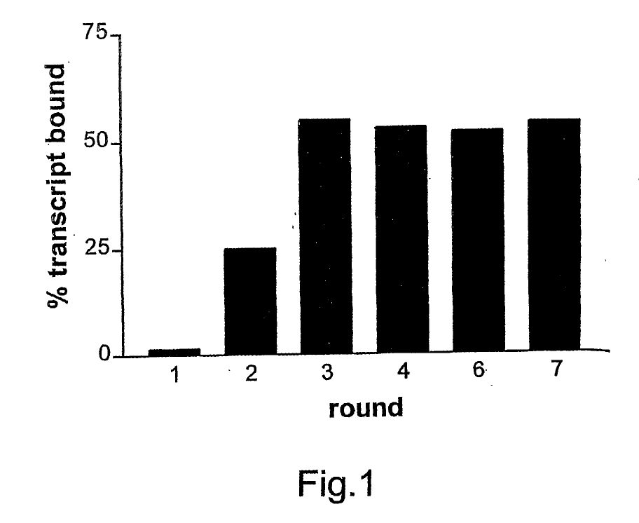

A DNA library comprising a 50 nucleotide randomized region was synthesized. In theory, this library could comprise 4⁵⁰=3×10²⁹ distinct sequences although in practice, only approximately 10¹⁴ of these are sampled during the selection process¹⁷. The library was transcribed to produce 2′ F-substituted RNA and subjected to repeated cycles of in vitro selection. Enrichment of PrP^{Sc}-specific nucleic acids was detected by measuring the depletion of nucleic acids from the supernatant during the partitioning step of successive rounds of selection. The results showed that PrP-binding nucleic acids became a significant fraction of the population by selection round 3 (FIG. 1). This pool was subjected to a further three rounds of selection although there appeared not to be further enrichment for PrPSc-binding aptamers (see FIG. 1). The pool of 2′ F-substituted RNA from round 7 was cloned as cDNA.

Affinity and Specificity of Monoclonal Aptamers for Recombinant Bovine PrP

Because we were interested in isolating aptamers that would be able to analyse PrP isolated from multiple species, we screened the in vitro-transcribed, monoclonal sequences against recombinant bovine PrP. We found that they all bound to bovine PrP in a concentration-dependent manner (see, for example, aptamer 73 at FIG. 2A) and displayed single-site binding characteristics with K_{D} in the 200-800 nM range (see FIG. 2B).

In order to determine the degree of cross-reactivity between natural and recombinant PrPs of different species, we performed band-shift assays using recombinant bovine, ovine and murine PrP (see FIG. 2C, for aptamer 93). The results show that all of the PrP-specific aptamers react with all forms of PrP from diverse species. In order to check that the aptamers did not have a general ability to bind proteins, we performed analogous band-shifts using recombinant human CD4 and streptavidin, both of which have the ability to generate aptamers²⁹ and Tahiri-Alaoui and James (unpublished results). The results (FIG. 2C lanes 9 and 10) show that the binding of the aptamers is PrP-specific.

As a final confirmation of specificity, we were able to show that unlabeled aptamer ap90 was able to displace end-labeled ap90 from PrP-aptamer complexes in a concentration-dependent manner that indicates an affinity in the order of approximately 100 nM (see FIG. 2D)

Conversion of Recombinant Bovine PrP to βrich Form in vitro

In order to assess whether the binding of PrP-specific aptamers was affected by the conformation of the protein, we needed a standard preparation of pure, monomeric and soluble PrP of the β-form. Accordingly, we denatured and reduced the disulphide bridges of the native, α-form, recombinant bovine PrP and refolded it under low pH conditions, largely as described by Jackson et al.³⁰. The resultant protein had lost the characteristic CD spectrum of α-form PrP and had a spectrum consistent with that of β-form PrP (FIG. 3A). This is different from previous reports in three respects. First, we used a full-length PrP and not a truncation, like 90-231³⁰. Second, we used bovine PrP, rather than human or hamster PrP. Finally, refolding was done at pH5, rather than pH4. This β-form of the protein was found not to be degraded or otherwise covalently modified when analysed by SDS-PAGE (FIG. 3B) or electro-spray mass spectrometry (data not shown). However, the β-form PrP had a lower mobility on a native gel system (FIG. 3C), perhaps indicative of a change in conformation from the a form to a more extended β form.

Differential Affinity of Aptamers for α-form and β-Form Bovine PrP

The titration of ³²P-labelled aptamer and 13-PrP was monitored using band-shift assays as described before. Titration of β-PrP into labeled aptamer at a concentration of approximately 60 nM yields a complex of slower electrophoretic mobility than the unbound aptamer as shown in FIG. 4A. To determine the apparent affinity constant for this aptamer β-PrP interaction, the amount of ³²P present in the free and bound aptamer bands was quantified and the binding data were fitted by non-linear regression to a hyperbolic function (FIG. 4B). This gave values of 74(±3) nM, 25(±5) nM, 37(±2) nM and 22(±5) nM for aptamers 90 and 93 respectively. These dissociation constants are between 10 and 20-fold lower than those determined for the α-form of bovine PrP, hence the aptamers bound to β-PrP with substantially higher affinity than to α-PrP (see FIG. 4C). Interestingly, the aptamer-β-PrP complex has a faster electrophoretic mobility than the complex between the same aptamers and α-PrP.

Discrimination Between Normal and Abnormal Forms of PrP by Aptamers

We performed a band-shift assay in which we mixed a PrP-specific aptamer with equimolar mixtures of the α and β forms at a range of concentrations to see whether the differential affinity of aptamers for α-form and β-form PrP could be used to discriminate between the two isoforms present in the same sample (FIG. 6A). The results show that the band corresponding to β PrP-aptamer complexes appeared at lower concentrations of the PrP mix than did the band corresponding to aptamer-α PrP complexes.

Next, we tested whether the observed difference in affinity of aptamer 73 for α and β-forms of recombinant PrP could provide the basis for screening samples of animal brain for the presence of TSE material. Accordingly, we took samples of brain from TSE-infected and control cattle, mice and hamsters, and of PrP^{0/0} null mice and analysed the Sarcosyl-insoluble, proteinase K-resistant fraction of each by gel mobility shift assay using aptamer 73 (FIG. 5B). Samples from TSE-infected animals of all three species are characterized by the presence of two bands: a band of mobility similar to that of the RNA-β-form complex seen in previous experiments and a protease-resistant aggregate that fails to enter the gel (FIG. 5B). Samples from uninfected cattle, mouse and hamster brains do not produce either band and, significantly, neither does that from the PrP-null mouse (FIG. 5B). When parallel samples were blotted onto nitrocellulose and probed with PrP-specific antibody 6H4 the aggregate was shown to contain PrP (FIG. 5B). Finally, we examined crude human brain homogenates by a similar method. Following proteinase K-treatment, high molecular weight complexes of PrP and aptamers only formed with brain homogenates prepared from individuals with sporadic and variant CJD and not with homogenate of normal human brain (FIG. 5C).

Sequences of PrP-Binding Aptamers

The sequences of fifty clones encoding ligands for PrP (FIG. 6) show that the great majority of ligands fell into a closely related group, possibly deriving from a single sequence present in the initial library, although insertion, deletion and substitution of nucleotides during in vitro evolution produced divergence of up to 25% between members of the group. Six other PrP-binding sequences were identified, each of which probably derived from a distinct member of the initial library. Intriguingly, these contained some stretches of weak sequence homology with the main group consensus and with each other that might reflect some convergence of structural features.

Secondary Structure and Epitope-Mapping of PrP-Binding Aptamers

In order to identify whether the sequence features identified above were related to the structure and function of the aptamers, we investigated their secondary structure. Structure-sensitive nucleases detect regions of single-strandedness (S1 and T1) or double-strandedness (V1). Chemical probing detects whether otherwise reactive groups are engaged in Watson-Crick H-bonds. In FIGS. 7A and B, we illustrate a study of one aptamer, clone 93, which had shown some sequence similarities with aptamers from Group I. In the 3′ half of the aptamer, both enzymatic and chemical probing methods were consistent with domains 3 and 4 in the secondary structure predicted using the stochastic algorithm of STAR software^{31,32} (FIG. 7D,). However, the 14nt at the 5′ end of the randomized portion of the aptamer (nt 24-37; domain 2) and a portion of the 5′ fixed sequence (nt 10-16; within domain 1) gave patterns of nuclease sensitivity consistent with a more double-stranded structure than that predicted by software.

Interestingly, when enzymatic probing was done in the presence of increasing amounts of soluble recombinant alpha-form of bovine PrP (FIG. 7C), these portions of the 5′ half of the molecule showed the greatest protection from nuclease attack, suggesting they contained the binding site for PrP (FIG. 7E). Further, the region of weak homology with group 1 aptamers coincided with this region, suggesting that the evolutionary convergence was adaptively significant.

Discussion

Here we describe the isolation of novel nucleic acid ligands for PrP, the key protein in the pathogenesis and transmission of vCJD, BSE and all other TSEs. Whereas previously described nucleic acid ligands, or aptamers, for PrP were composed of nuclease-sensitive RNA, the aptamers described here are composed of nucleaseresistant, 2′ F-substituted nucleic acid, providing a significant advantage when studying nuclease-rich samples, such as the brain. Moreover, unlike previous aptamers, those we describe here have substantially higher affinity for the β-form of PrP than for its α-isoform. Although one monoclonal antibody has been described that has a greater affinity for aggregated PrP compared to the normal isoform of the protein it is not widely available and, unlike the aptamers described here, is sensitive to proteases.

The great majority of the PrP-binding sequences described here are so closely related that they appear to be derived from a single, ancestral library sequence. The 5′ half of this group is predicted to fold into two helix-loop domains separated by an 8nt unstructured region (see FIG. 7). Minor sequence variation between members of this group preserves base-pairing within the two helices, and enzymatic probing confirms that they are, indeed, double-stranded. The region between the first two helices, which is predicted to be unstructured, shows sensitivity to VI endoribonuclease, suggesting substantial base-stacking, involvement in tertiary structure elements or non-canonical base-pairs. Six PrP-binding aptamers, whose sequence shows that they clearly derive from distinct members of the starting library, nevertheless show patches of homology with the main group around this putatively unstructured region. Moreover, in each case, the region of homology is predicted to be unstructured and shows paradoxical V1 reactivity. Significantly, in each case, this region is the focus of nuclease protection in the presence of PrP, suggesting that it comprises the contact site for the target protein. Consequently, it is most probable that the-structural motif responsible for PrP binding is homologous in all the aptamers here described. Studies of this sort cannot give definitive structural data, but we suggest that the most likely common PrP-binding motif is:

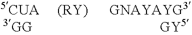

The basis for the preferential recognition of β-form PrP by these aptamers may be revealed by the gel-shift experiments. First, we should note that in the absence of aptamer, the β-form PrP migrates more slowly than the native form in non-denaturing gels. This is expected if the β-rich structure were to adopt a more extended structure, with a consequent increase in solvent-exposed surface, upon transformation from the globular, α helix-rich native form. However, we saw that the mobility of the complex formed between aptamer and the β-form of PrP was greater than that with the α-form, suggesting that the solvent-exposed surface of the former complex was less than that of the latter. This suggests that the area of contact between aptamer and the β-form is greater than with the α-form, and this is consistent with a higher affinity for β than α. It has previously been reported that RNA interactions with proteins are generally more favorable with β-sheet than α helix³³.

We also see evidence that the contact region of aptamer becomes more structured after interaction with PrP, as evidenced by increases in V1 reactivity. The increased electrophoretic mobility of the aptamer-β PrP complex may also be attributed to a dramatic change in the conformation of the bound aptamer. This gel mobility enhancement has also been observed with the RNA binding protein HIV Rev and alfalfa mosaic virus coat protein^{34,35}. These interactions should provide us with tools for studying the conformational transitions believed to occur to PrP during the pathogenesis of scrapie, BSE and CJD.

We have been able to show that the differential affinity of these aptamers for the β-form of PrP, together with their resistance to proteases and nucleases, enables one to detect disease-associated form of PrP in the brains of infected cattle, mouse and hamsters. Even more strikingly, the same aptamer was able to differentiate between crude brain samples from normal and CDJ-affected humans. While gel-shift assays are probably impractical for routine use, the results indicate that 2′ F RNA aptamers could be used in order to develop a more reliable and sensitive test for sub-clinical infection with TSEs such as BSE and vCJD than is presently available.

References

The following documents have been referred to, and are incorporated herein by reference.

1. Hope, J. et al. The major polypeptide of scrapie-associated fibrils (SAF) has the same size, charge distribution and N-terminal protein sequence as predicted for the normal brain protein (PrP). Embo J5, 2591-7 (1986).

2. Prusiner, S. B. Prions. Proc. Natl. Acad. Sci. USA 95, 13363-13383 (1998).

3. Riek, R. et al. NMR structure of the mouse prion protein domain PrP(121-321). Nature 382, 180-2 (1996).

4. James, T. L. et al. Solution structure of a 142-residue recombinant prion protein corresponding to the infectious fragment of the scrapie isoform. Proc Natl Acad Sci USA 94, 10086-91 (1997).

5. Zahn, R. et al. NMR solution structure of the human prion protein. Proc Natl Acad Sci USA 97, 145-50 (2000).

6. Zhang, H. et al. Physical studies of conformational plasticity in a recombinant prion protein. Biochemistry 36, 3 543-53 (1997).

7. Kocisko, D. A., Lansbury Pt, Jr. & Caughey, B. Partial unfolding and refolding of scrapie-associated prion protein: Evidence for a critical 16-kDa C-terminal domain. Biochemistry 35, 13434-13442 (1996).

8. Wildegger, G., Liemaun, S. & Glockshuber, R. Extremely rapid folding of the C-terminal domain of the prion protein without kinetic intermediates. Nat Struct Biol 6, 5 50-3 (1999).

9. Fersht, A. R. Transition-state structure as a unifying basis in protein-folding mechanisms: Contact order, chain topology, stability, and the extended nucleus mechanism. Proceedings of the National Academy of Sciences of the United States of America 97, 1525-1529 (2000).

10. Krasemann, S., Groschup, M. H., Harmeyer, S., Hunsmann, G. & Bodemer, W. Generation of monoclonal antibodies against human prion proteins in PrP(0/0) mice. Molecular Medicine 2, 725-734 (1996).

11. Williamson, R. A. et al. Circumventing tolerance to generate autologous monoclonal antibodies to the prion protein. Proceedings of the National Academy of Sciences of the United States of America 93, 7279-7282 (1996).

12. Korth, C. et al. Prion (PrPSc)-specific epitope defined by a monoclonal antibody. Nature 390, 74-7 (1997).

13. Meyer, R. K., Oesch, B., Fatzer, K., Zurbriggen, A. & Vandevelde, M. Detection of bovine spongiform encephalopathy-specific PrP(Sc) by treatment with heat and guanidine thiocyanate. J Virol 73, 93 86-92 (1999).

14. Weiss, S. et al. RNA aptamers specifically interact with the prion protein PrP. J Virol 71, 8790-7 (1997).

14a. WO 9743649. Chaperones capable of binding to prion proteins and distinguishing the isoforms PrP^{C} and PrP^{SC}.

15. Williams, D. M., Benseler, F. & Eckstein, F. Properties of 2′-fluorothymidine-containing oligonucleotides: interaction with restriction endonuclease EcoRV. Biochemistry 30, 4001-9 (1991).

16. Pieken, W. A., Olsen, D. B., Benseler, F., Aurup, H. & Eckstein, F. Kinetic characterization of ribonuclease-resistant 2′-modified hammerhead ribozymes. Science 253, 314-7 (1991).

17. Tuerk, C. Using the SELEX combinatorial chemistry process to find high affinity nucleic acid ligands to target molecules. Methods Mol Biol 67, 2 19-30 (1997).

18. Heidenreich, O. et al. Ribozyme-mediated RNA degradation in nuclei suspension. Nucleic Acids Res 23, 2223-8 (1995).

19. Kimberlin, R. H. & Walker, C. A. Evidence that the transmission of one source of scrapie agent to hamsters involves separation of agent strains from a mixture. J Gen Virol 39, 487-96 (1978).

20. Adams, T. E., MacIntosh, B., Brandon, M. R., Wordsworth, P. & Puri, N. K. Production of methionyl-minus ovine growth hormone in Escherichia coli and one-step purification. Gene 122, 371-5 (1992).

21. Felden, B., Florentz, C., Giege, R. & Westhof, E. Solution structure of the 3′-end of brome mosaic virus genomic RNAs. Conformational mimicry with canonical tRNAs. J Mol Biol 235, 508-3 1 (1994).

22. Aurup, H., Williams, D. M. & Eckstein, F. 2′-Fluoro- and 2′-amino-2′-deoxynucleoside 5′˜triphosphates as substrates for T7 RNA polymerase. Biochemistry 31, 9636-41 (1992).

23. Peattie, D. A. & Gilbert, W. Chemical probes for higher-order structure in RNA. Proc Natl Acad Sci USA 77, 4679-82 (1980).

24. Ehresmann, C. et al. Probing the structure of RNAs in solution. Nucleic Acids Res 15, 9 109-28 (1987).

25. Stern, S., Moazed, D. & Noller, H. F. Structural analysis of RNA using chemical and enzymatic probing monitored by primer extension. Methods Enzymol 164, 481-9 (1988).

26. Sanger, F., Nicklen, S. & Coulson, A. R. DNA sequencing with chain-terminating inhibitors. Proc Natl Acad Sci USA 74, 5463-7 (1977).

27. Jackson, G. S. et al. Reversible conversion of monomeric human prion protein between native and fibrilogenic conformations. Science 283,1935-7 (1999).

28. Reisfeld, R. A., Lewis, V. J. & Williams, D. E. Disc Electrophoresis of Basic Proteins and Peptides on Polyacrylamide Gels. Nature 195, 281-283 (1962).

29. Kraus, E., James, W. & Barclay, A. N. Novel RNA ligands able to bind CD4 antigen and inhibit CD4+ T lymphocyte function. Jlmmunol 160, 5209-12 (1998).

30. Jackson, G. S. et al. Multiple folding pathways for heterologously expressed human prion protein. Biochim Biophys Acta 1431, 1-13 (1999).

31. Gultyaev, A. P. The computer simulation of RNA folding involving pseudoknot formation. Nucleic Acids Res 19, 2489-94 (1991).

32. Abrahams, J. P., van den Berg, M., van Batenburg, E. & Pleij, C. Prediction of RNA secondary structure, including pseudoknofting, by computer simulation. Nucleic Acids Res 18, 303 5-44 (1990).

33. Steitz, T. A. RNA Recognition by Proteins, in The RNA World (ed. Raymond F. Gesteland, T.R.d.a.J.F.A.) 427-450 (dold Spring Harbor Laboratory, New York, 1999).

34. Kjems, J., dalnan, B. J., Frankel, A. D. & Sharp, P. A. Specific binding of a basic peptide from HIV-1 Rev. Embo J, 1119-29 (1992).

35. Baer, M. L., Houser, F., Loesch-Fries, L. S. & Gehrke, L. Specific RNA binding by amino-terminal peptides of alfalfa mosaic virus coat protein. Embo J 13, 727-3 5 (1994).

1. A ligand for PrP which is protease resistant and nuclease resistant.

2. A ligand according to claim 1 which binds to at least one isoform of PrP with an affinity constant in the range of 1 to 10,000 nM.

3. A ligand for PrP which is selective for a PrP isoform.

4. A ligand according to claim 1 or 3 which is selective for PrP^{Sc}.

5. A ligand according to claim 4, where the ratio of affinity constants for PrP^{C}:PrP^{Sc} is at least 5:1.

6. A ligand for PrP which is a nuclease-resistant aptamer.

7. A ligand according to claim 6 which is a 2′-F-substituted nucleic acid

8. A ligand according to claim 7, which incorporates the structural motif:
^{5′}CUA(RY)GNAYAYG3′
^{3′}GG GY^{5′}

9. A method for detecting a PrP which comprises contacting a sample with a ligand according to any preceding claim and determining if there is ligand-PrP binding.

10. A method for detecting a TSE which comprises contacting a sample with a ligand according to claim 4 and determining if there is ligand-PrP^{Sc} binding.
